(19) United States
(12) Patent Application Publication
(10) Pub. No.: US 2011/0123609 A1
(43) Pub. Date: May 26, 2011

(54) MULTIPLE UNIT DOSAGE FORM OF NIACIN

(76) Inventors: Sunil Shantwan Borude, Maharashtra (IN); Shrenik Annasaheb Kole, Maharashtra (IN); Makarand Krishnakumar Avachat, Maharashtra (IN)

(21) Appl. No.: 13/054,901

(22) PCT Filed: Jul. 16, 2009
(86) PCT No.: PCT/IN09/00404

§ 371 Date: Jan. 19, 2011

(51) Int. Cl. A61K 9/48; A61K 31/455; A61P 3/06

(52) U.S. Cl. 424/452; 514/356

(57) ABSTRACT

A multiple unit dosage form useful for treating or preventing hyperlipidemia and/or atherosclerosis, wherein multiple unit dosage form comprise of a therapeutically effective amount of niacin or its derivatives and one or more control releasing agent(s) and pharmaceutically acceptable excipients, weight percentages are based upon the total weight of the dosage form. The multiple unit dosage form may comprise of optionally other antihyperlipidemic agent, more preferably HMG CoA reductase inhibitor. The most preferable dosage form is capsule. Further a kit comprising one or more capsules co-packaged to provide multiple unit dosage form of niacin or its derivatives in combination with HMG CoA reductase inhibitor is disclosed.

### FIELD OF INVENTION

The present invention relates to a multiple unit dosage form of niacin or its derivatives comprising niacin or its derivatives and control releasing agent(s). The present invention also relates to a multiple unit dosage forms comprising niacin or its derivatives in combination with HMG CoA reductase inhibitor wherein niacin or its derivative is present in controlled release form and HMG CoA reductase inhibitor is present in immediate release form.

### BACKGROUND OF INVENTION

Hyperlipidemia or an elevation in serum lipids is associated with an increase incidence of cardiovascular disease and atherosclerosis. It is known that the likelihood of cardiovascular disease can be decreased, if the serum lipids, and in particular low-density lipoprotein-cholesterol (LDL-cholesterol), can be reduced. Further, the progression of atherosclerosis can be retarded or the regression of atherosclerosis can be induced if serum lipids can be lowered.

In such cases, individuals diagnosed with hyperlipidemia are prescribed lipid-lowering therapy for the purposes of reducing their risk of cardiovascular disease.

People with hyperlipidemia also have elevated triglyceride levels. It is known that a reduction in elevated triglycerides can result in the secondary lowering of cholesterol. These individuals should also consider lipid-lowering therapy to reduce their elevated triglycerides for purposes of decreasing their incidence of atherosclerosis and coronary artery disease.

Low-density lipoprotein carries cholesterol in the blood to the subendothelial spaces of blood vessel walls. It is believed that peroxidation of LDL-cholesterol within the subendothelial space of blood vessel walls leads to atherosclerosis plaque formation.

High-density lipoprotein (HDL-cholesterol), on the other hand, is believed to counter plaque formation and delay or prevent the onset of cardiovascular disease and atherosclerotic symptoms.

In the past, there have been numerous methods proposed for reducing elevated cholesterol levels and for increasing HDL-cholesterol levels for the purpose of treating hyperlipidemia and arteriosclerosis or cardiovascular diseases.

Typical drugs for the treatment of hyperlipidemia or hypercholesterolemia include inhibitors of HMG-CoA reductase, the rate-controlling enzyme in the biosynthetic pathway of cholesterol. Examples of HMG-CoA reductase inhibitors include but not limited to mevastatin, lovastatin, pitavastatin, velostatin, simvastatin, rivatatin, fluvastatin, atorvastatin and cerivastatin.

Other antihyperlipidemic agents include but not limited to niacin or its derivatives, bile acid sequestrants such as cholestyramine, colestipol DEAE-Sephadex, probucol, and related compounds, as well as lipostabil, Eisai E-5050, imanixil, tetrahydrolipstatin, istigmastanyl phosphorylcholine, aminocyclodextrin, Ajinomoto AJ-814, melinamide, Sandoz 58-035, American Cyanamid CL-277,082 and CL-283,546, ronitol, neomycin, p-aminosalicylic acid, aspirin, quaternary amine poly(diallyldimethylammonium chloride) and ionenes, poly(diallylmethylamine) derivatives, omega-3-fatty acids found in various fish oil supplements and fibric acid derivatives. Further the combination of different anti-hyperlipidemic agents for the treatment of hyperlipidemia is also possible.

Niacin or nicotinic acid has been used for many years in the treatment of hyperlipidemia or hypercholesteremia. This compound has been known to exhibit the beneficial effects of reducing total cholesterol, very low-density lipoprotein (VLDL-cholesterol) and VLDL-cholesterol remnants, LDL-cholesterol, triglycerides and apolipoprotein, while increasing desirable HDL-cholesterol.

Fast release niacin or its derivatives has conventionally been administered three times per day after meals, but cutaneous flushing often occurs in the hyperlipidemics to whom the niacin or its derivative is administered.

In order to avoid or alleviate the cutaneous flushing resulting from niacin or its derivatives therapy, a number of agents have been suggested. Another method of avoiding or reducing the side effects associated with fast release niacin is the use of extended or sustained release formulations. Extended or sustained release formulations are designed to slowly release the active ingredient from the tablet, which allows a reduction in dosing frequency as compared to the typical dosing frequency associated with conventional or fast dosage forms. The slow drug release reduces and prolongs blood levels of the drug and, thus, minimizes or lessens the cutaneous flushing side effects that are associated with conventional or fast release niacin products.

U.S. Pat. No. 5,126,145 discloses controlled release uncoated tablet comprising water soluble active ingredient, hydroxypropyl methylcellulose, polyvinyl pyrrolidone, and vegetable oil or stearic acid.

U.S. Pat. No. 6,080,428 discloses a method of treating hyperlipidemia using an oral solid dosage form that consists essentially of nicotinic acid, hydroxypropyl methylcellulose, a water-soluble binder and a lubricant

US Appl. no. 20070237819 discloses sustained release oral solid dosage form comprising niacin or nicotinic acid, hydroxypropyl methylcellulose, binder and lubricant.

US Appl. no. 20070264332 discloses sustained/prolonged release pharmaceutical composition comprising niacin, a polymer mixture comprising polyvinyl acetate and polyvinyl pyrrolidone combined with cellulose ether polymer.

U.S. Pat. No. 6,491,950 discloses sustained release capsule comprising niacin or niacin derivatives thereof, high melting fatty acid ester, oil, cellulosic polymer and surfactant.

U.S. Pat. No. 6,469,035 discloses a method of altering serum lipid levels by orally administering an oral solid dosage form comprised of an effective lipid-altering amount of the extended release nicotinic acid and an effective lipid-altering amount of an immediate release HMG-CoA reductase inhibitor. Further pretreatment with flush inhibiting agent is also disclosed to reduce the flushing.

Sustained release oral tablets of niacin, Niaspan®, are marketed by Abbott and are approved for treating hyperlipidemia. Further the combination of niacin in combination with lovastatin and niacin in combination with simvastatin is marketed as Advicor® and Simcor®, respectively by Abbott. Niaspan is indicated as an adjunct to diet for reduction of elevated TC, LDL-C, Apo B and TG levels, and to increase HDL-C in patients with primary hypercholesterolemia and mixed dyslipidemia. In combination with lovastatin or simvastatin it is used in the treatment of primary hypercholesterolemia and mixed dyslipidemia. In patients with a history of myocardial infarction and hypercholesterolemia, niacin is indicated to reduce the risk of recurrent nonfatal myocardial infarction. In patients with a history of coronary artery disease (CAD) and hypercholesterolemia, niacin, in combination with a bile acid binding resin, is indicated to slow progression or promote regression of atherosclerotic disease. Niaspan in combination with a bile acid binding resin is indicated as an adjunct to diet for reduction of elevated TC and LDL-C levels in adult patients with primary hypercholesterolemia when the response to an appropriate diet, or diet plus monotherapy, has been inadequate. It is also indicated as adjunctive therapy for treatment of adult patients with very high serum triglyceride levels who present a risk of pancreatitis and who do not respond adequately to a determined dietary effort to control them.

We have developed multiple unit dosage form of niacin in an effort to make an alternate dosage form, a capsule dosage form, comprising of a combination of niacin in extended release form and optionally HMG CoA reductase inhibitor in immediate release form for those individuals that either have difficulty in swallowing a tablet or who prefer a capsule dosage form.

The present invention describes an oral capsule dosage form of niacin or its derivatives, prepared in relatively few steps. Further, capsules have several advantages. For example, capsules ensure accurate dosing since they cannot be split. This helps eliminate any confusion about the proper dosage, especially for the elderly patients. This regimen can help to improve patient compliance and thus effective pharmacotherapy. Also, capsule-manufacturing process is generally simpler and requires less equipment.

In addition, capsule offers advantages including a) free dispersion of controlled release of niacin from capsule in the GI tract, providing a steady drug release into blood stream, b) minimum side effects and c) reduction of inter- and intra-patient variability.

### OBJECTIVE OF THE INVENTION

The object of present invention is to provide a multiple unit dosage form comprising at least or more than about 50% of a therapeutically effective amount of antihyperlipidemic agent and one or more control releasing agent(s), weight percentages are based upon the total weight of the dosage form. The multiple unit dosage form may optionally contain other anti-hyperlipidemic agent.

Another object of the present invention is to provide a multiple unit dosage form comprising at least or more than about 50% of a therapeutically effective amount of niacin or its derivatives and control releasing agent(s), wherein the control releasing agent is used in combination of hydrophobic and/or hydrophilic agent in the ratio of 1:50 to 50:1, weight percentages are based upon the total weight of the dosage form.

Another object of the present invention is to provide a multiple unit dosage form comprising at least or more than about 65% of a therapeutically effective amount of niacin or its derivatives, one or more control releasing agent(s) filled in capsule of suitable size, weight percentages are based upon the total weight of the dosage form.

Another object of the present invention is to provide a multiple unit dosage form comprising at least or more than about 65% of a therapeutically effective amount of niacin or its derivatives coated with one or more control release agent(s) wherein the percent weight gain of coating is from about 2 to about 20%, filled in capsule of suitable size, weight percentages are based upon the total weight of the dosage form.

Another object of the present invention is to provide a kit for multiple unit dosage form of niacin or its derivatives comprising one or more capsules co-packaged to provide a dosage up to 2000 mg of niacin. Further HMG CoA reductase inhibitor can be optionally present.

Another object of the present invention is to provide a multiple unit dosage form comprising of a therapeutically effective amount of niacin in combination with HMG CoA reducatse inhibitor, optionally coated and filled in capsule of suitable size.

### SUMMARY OF THE INVENTION

Accordingly one aspect of the present invention is to provide a multiple unit dosage form comprising at least or more than about 50% of a therapeutically effective amount of antihyperlipidemic agent(s) and one or more control releasing agent(s), weight percentages are based upon the total weight of the dosage form. The multiple unit dosage form may optionally contain other anti-hyperlipidemic agent.

Another embodiment of the present invention is to provide a multiple unit dosage form comprising at least or more than about 50% of a therapeutically effective amount of niacin or its derivatives and control releasing agent(s), wherein the control releasing agent is used in combination of hydrophobic and/or hydrophilic agent in the ratio of 1:50 to 50:1, weight percentages are based upon the total weight of the dosage form.

Another embodiment of the present invention is to provide a multiple unit dosage form comprising at least or more than about 65% of a therapeutically effective amount of niacin or its derivatives, one or more control releasing agent(s) filled in capsule of suitable size, weight percentages are based upon the total weight of the dosage form.

Another embodiment of the present invention is to provide a multiple unit dosage form comprising at least or more than about 65% of a therapeutically effective amount of niacin or its derivatives coated with one or more control release agent(s) wherein the percent weight gain of coating is from about 2 to about 20%, filled in capsule of suitable size, weight percentages are based upon the total weight of the dosage form.

Another embodiment of the present invention is to provide a kit for multiple unit dosage form of niacin or its derivatives comprising one or more capsules co-packaged to provide a dosage up to 2000 mg of niacin. Further HMG CoA reductase inhibitor can be optionally present.

Another object of the present invention is to provide a multiple unit dosage form comprise of a therapeutically effective amount of niacin in combination with HMG CoA reducatse inhibitor, optionally coated and filled in capsule of suitable size.

### DETAILED DESCRIPTION OF THE INVENTION

The present invention relates to the multiple unit dosage form of the present invention comprising antihyperlipidemic agent(s) and control releasing agent(s). More preferably the multiple unit dosage form of the present invention comprises of anti-hyperlipidemic agent(s) and control releasing agent(s) filled in capsule. Furthermore the preferred anti-hyperlipidemic agent is niacin or nicotinic acid or HMG CoA reductase inhibitor.

The terms controlled release, sustained release, sustained action, prolonged action, extended action; extended release, timed release, pulsatile release etc. are used interchangeably in the present invention.

The anti-hyperlipidemic agents used in the present invention includes but are not limited to niacin or its derivative, HMG Co-A reductase inhibitor as mevastatin, lovastatin, pitavastatin, velostatin, simvastatin, rivastatin, fluvastatin, rosuvastatin, atorvastatin and cerivastatin, bile acid sequestrants such as cholestyramine, colestipol DEAE-Sephadex, probucol, and related compounds, as well as lipostabil, Eisai E-5050, imanixil, tetrahydrolipstatin, istigmastanylphospho-rylcholine, aminocyclodextrin, Ajinomoto AJ-814, melinamide, Sandoz 58-035, American Cyanamid CL-277,082 and CL-283,546, ronitol, neomycin, p-aminosalicylic acid, aspirin, quaternary amine poly(diallyldimethylammonium chloride) and ionenes, poly(diallylmethylamine) derivatives, omega-3-fatty acids found in various fish oil supplements and fibric acid derivatives. Combination of antihyperlipidemic agent(s) is also included in the scope of the invention. The most preferred combination is niacin or its derivatives and HMG CoA reductase inhibitor.

Niacin and nicotinic acid is used interchangeably in the present invention. The amount of niacin or its derivatives in a controlled release dosage form of present invention is from about 250 to about 3000 mg.

Niacin or its derivatives of the present invention specifically include but not limited to the following: niacin or nicotinic acid, nicotinyl alcohol tartrate, D-glucitol hexanicotinate, aluminium nicotinate, niceritrol, D-L-alpha-tocopheryl nicotinate, 6-OH-nicotinic acid, nicotinaria acid, nicotinamide, nicotinamide-N-oxide, 6-OH-nicotinamide, NAD, N-methyl-2-pyridine-8-carboxamide, N-methyl-nicotinamide, N-ribosyl-2-pyridone-5-carboxide, N-methyl-4-pyridone-5-carboxamide, bradilian, sorbinicate, hexanicite, ronitol, and esters of nicotinic acid such as lower alcohol esters like methyl, ethyl, propyl or butyl esters. Each of such derivatives or compounds will be collectively referred to hereinabove by “niacin derivatives.”

The present invention thus involves a multiple unit dosage form of niacin or its derivatives, and control releasing agent(s), optionally comprising HMG CoA reductase inhibitor.

The present invention also involves a multiple unit dosage form comprising niacin or its derivatives and HMG Co-A reductase inhibitor. Preferably the niacin or its derivatives is in controlled release form and HMG CoA reductase inhibitor is in immediate release form. The HMG Co-A reductase inhibitor is selected from but not limited to mevastatin, lovastatin, pitavastatin, velostatin, simvastatin, rivastatin, fluvastatin, atorvastatin and cerivastatin. The most preferred HMG Co-A reductase inhibitor is lovastatin or simvastatin.

The multiple unit according to present invention includes but not limited to mini-tablets, tablets, pellets, spheroids, granules, beads or beadlets, discrete particles, microparticles, powder or any combination thereof. The multiple unit may be present in coated or uncoated form.

The preferred dosage form of the present invention is capsule filled with mini-tablets, tablet, pellets, spheroids, granules, beads or beadlets, discrete particles, microparticles, powder or any combination thereof. Preferably the dosage form of the present invention are coated with control releasing agent.

The present invention comprise of a multiple unit dosage form comprising at least more than about 50% of niacin or its derivatives. Preferably the multiple unit dosage form comprise of about more than 65% of niacin or its derivatives. Further the multiple unit dosage form optionally contains HMG CoA reductase.

Control releasing agent used in the present invention includes, hydrophilic agent, hydrophobic agent and combination thereof. Preferably the control release agent (s) used in the present invention comprise of a combination of hydrophilic agent and hydrophobic agent.

The ratio of hydrophilic and hydrophobic agent for the present invention is 1:50 to 50:1. More preferable the ratio between hydrophilic agent and hydrophobic agent is 1:25 to 25:1 and the most preferable ratio is 1:10 to 10:1.

Hydrophilic agent is selected from the group but not limited to cellulose derivatives, such as hydroxypropyl methylcellulose, sodium carboxy methyl cellulose, methyl cellulose, hydroxyethyl cellulose, hydroxypropyl cellulose, hydroxyethyl methylcellulose, hydroxyethylethyl cellulose, hydroxypropylethyl cellulose, alkyl hydroxypropyl methyl cellulose; polysaccharide family, such as corn starch, potato starch, alpha starch and hydroxyethyl starch as its derivative, dextrin and dextran as its derivative, maltodextrin, polydextrose, alginic acid alkali metal salt as a family of alginic acid; gum family, such as guar gum, locust bean gum, xanthan gum, cyclodextrin, arabic gum, gellan gum, karaya gum, casein, tara gum, tamarind gum, tragacanth gum and ghatti gum; peptide series, such as gelatin, collagen, protamine and zein; acrylic acid series carbomer, polyacrylamide, and other materials (e.g., polyvinylacetal diethylaminoacetate, glucomannan, glucosamine, arabinogalactane, furcelleran, pullulan, polyurethane, chitosan, chitin, agar, pectin and carrageenan, polyvinyl acetate, polyvinyl alcohol, povidone, polyethylene oxide and mixtures thereof.

Hydrophobic agent is selected from the group but not limited to water-insoluble cellulose derivative is ethyl cellulose or cellulose acetate, water-insoluble vinyl derivative is poly-vinyl acetate or polyvinyl chloride, paraffin and the stearate salts such as calcium, magnesium, zinc and mixtures thereof. acrylic polymers include, but are not limited to acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid, methacrylic acid alkylamine copolymer, poly(methyl methacrylate), poly(methacrylic acid) (anhydride), methyl methacrylate, polymethacrylate, poly(methyl methacrylate), poly(methyl methacrylate) copolymer, polyacrylamide, poly(methacrylic acid anhydride), and glycidyl methacrylate copolymers, waxes and mixtures thereof.

Wax is selected from the group but not limited to bees wax, carnuba wax, fatty acids such as stearic acid, capric acid, lauric acid, myristic acid, palmitic acid, undecanoic acid, caproic acid, caprylic acid, arachidic acid, behenic acid, oleic acid, linoleic acid or linolenic acid, long chain fatty alcohols such as lauryl alcohol, myristyl alcohol, cetyl alcohol, stearyl alcohol, undecanol, glycerides such as glyceryl esters of fatty acids like stearin, myristin, palmitin, laurin glyceryl monostearate, glyceryl distearate, glyceryl esters of hydrogenated castor oil, mineral oil, hydrogenated vegetable oil, acetylated glycerides.

The multiple unit dosage form of the present invention may further comprise of pharmaceutically acceptable excipients used in the art including but not limited to fillers or diluents, binders, disintegrants, glidants, lubricants, surface active agents, film forming agents, stabilizers, chelating agent, preservatives, pore forming agent and antioxidant.

Filler or diluent is selected from the group but not limited to microcrystalline cellulose, lactose, sugars, starches, modified starch, mannitol, sorbitol and other polyols, dextrin, dextran and maltodextrin, calcium carbonate, calcium phosphate and/or hydrogen phosphate, sulphate.

Binder is selected from the group but not limited to lactose, starches, modified starch, dextrin, dextran and maltodextrin, microcrystalline cellulose, sugars, polyethylene glycols, hydroxypropyl cellulose, hydroxypropyl methylcellulose, ethyl cellulose, hydroxyethylcellulose, methyl cellulose, carboxy methylcellulose, croscarmellose sodium, gelatin, acacia gum, tragacanth, polyvinyl pyrrolidone, magnesium aluminium silicate.

Disintegrating agent is selected from the group but not limited to croscarmellose sodium, cross-linked polyvinylpyrrolidone, cross-linked carboxymethyl starch, different starches and microcrystalline cellulose, magnesium aluminium silicate, polyacrylin potassium.

Glidant is selected from the group but not limited to magnesium stearate, calcium stearate, zinc stearate, calcium behenate, sodium stearyl fumarate, talc, magnesium trisilicate, stearic acid, palmitic acid, carnauba wax, silicon dioxide.

Lubricant is selected from the group but not limited to magnesium stearate, other alkali earth metal stearate; colloidal anhydrous silica, talc, sodium stearyl fumarate, glyceryl monostearate and the like.

Surface-active agents and other conventional components for multiple unit dosage form can be included, such as colouring agents, lakes, aromas, opacifier and adsorbents. As a surface active agent the following may be used: ionic surfactants, such as sodium lauryl sulphate or non-ionic surfactants such as different poloxamers (polyoxyethylene and polyoxypropylene copolymers), natural or synthesized lecithins, esters of sorbitan and fatty acids (such as Span®. [Atlas Chemie]), esters of polyoxyethylenesorbitan and fatty acids (such as Tween®. [Atlas Chemie]), polyoxyethylated hydrogenated castor oil (such as Cremophor® [BASF]), polyoxyethylene stearates (such as Myrj®. [Atlas Chemie]) or any combination of the herein above mentioned surface active agents.

Film- or layer-forming agent is selected from the group but are not limited to cellulose family, such as noncrystalline cellulose, hydroxypropylmethyl cellulose, sodium carboxymethyl cellulose, methyl cellulose, ethyl cellulose, hydroxyethyl cellulose, hydroxypropyl cellulose, hydroxyethyl methyl cellulose, hydroxyethylethyl cellulose, hydroxypropyl ethyl cellulose, alkyl hydroxypropylmethyl cellulose, hydroxypropylmethyl cellulose phthalate, hydroxypropyl methyl cellulose acetate succinate, cellulose acetate and cellulose acetate phthalate; polysaccharide family, such as corn starch, potato starch and alpha starch and its derivative hydroxyethyl starch, dextrin and its derivative dextran, maltodextrin and polydextrose; gum family, such as guar gum, locust bean gum, xanthan gum, cyclodextrin, arabic gum, gellan gum, karaya gum, casein, tara gum, tamarind gum, tragacanth gum and ghatti gum; peptide family, such as gelatin, collagen, protamine and zein; acrylic acid family, such as carbomer and polyacrylamidewax family, such as carnauba wax and beeswax; carbohydrate family, such as sucrose, liquid sucrose, lactose and polyvinylacetal diethylaminoacetate and other materials (e.g., polyurethane, chitosan, chitin, agar, pectin, carrageenan and shellac).

The term “stabilizer” as used herein means an agent that stabilizes by any mechanism, for example alkanizing agent or buffering agent, chelating agent, photoprotectant and antioxidant.

The stabilizer may be selected from a group of alkalizing agent or buffering agent, chelating agent, photoprotectant and antioxidant or combination thereof.

Chelating agents is selected from but not limited to disodium EDTA, edetic acid, citric acid, and combinations thereof.

Photoprotectant is selected from but not limited to metal oxides such as titanium oxide, ferric oxide or zinc oxide or combination thereof.

The alkalizing or buffering agent is selected from a group but not limited to alkali metal salt, alkaline earth metal salt, oxides and hydroxides of alkaline and/or alkali-earth metals, ammonium salts of citric acid, ascorbic acid, maleic acid, sorbic acid, succinic acid, benzoic acid, phosphoric acid, carbonic acid, sulfuric acid, nitric acid, boric acid and silicic acid, ion exchangers, amines alone or in combination with a strong or weak acid, alkali amino acids, saccharides or combination thereof. Further other organic or inorganic alkalizing agents can be used.

Antioxidant is selected from the group but not limited to butylated hydroxyanisole (BHA), butylated hydroxytoluene (BHT), ascorbic acid, citric acid, malic acid, sodium ascorbate, sodium citrate, sodium metabisulphite, vitamin A, vitamin E, selenium, amino acids such as cysteine and methionine, or a combination thereof.

Another embodiment of the present invention includes a multiple unit dosage form comprising at least or more than about 50% of niacin or its derivatives and control-releasing agent(s), optionally comprising HMG CoA reductase inhibitors the multiple unit is filled in capsule. The capsule may be hard gelatin or soft gelatin capsule. Preferably the capsule shell is prepared from gelatin, starch, HPMC, plant derived material and the like.

Thus the present invention includes mini-tablets, tablets, pellets, spheroids, granules, beads or beadlets, discrete particles, microparticles, powder or any combination thereof comprising at least or more than about 50% of niacin or its derivatives and control releasing agent(s), optionally comprising HMG CoA reductase inhibitors, filled in capsule. More preferably, the present invention includes mini-tablets, pellets, spheroids, granules, beads or beadlets, discrete particles, microparticles, powder or any combination thereof comprising at least more than about 65% of niacin or its derivatives and a combination in the ratio of about 1:50 to 50:1 of hydrophilic and hydrophobic agent to control the release of niacin or its derivatives filled in capsule. Further the present invention comprise of at least or more than about 50% of niacin or its derivatives and optionally HMG CoA reductase inhibitor. The niacin or its derivatives is in controlled release form and HMG CoA reductase inhibitor may be in controlled release or immediate release form. Preferably the dosage form of the present invention is coated with control releasing agent.

Another embodiment of the present invention is in the form of a kit comprising one or more multiple unit dosage form comprising anti-hyperlipidemic agent, preferably niacin or its derivatives co-packaged to provide a dosage up to 3000 mg of niacin. In addition, a kit may comprise of multiple unit dosage form of niacin or its derivatives in combination with HMG CoA reductase inhibitor. The preferred multiple unit dosage form is in the form of capsule. Thus a kit includes one or more capsules of 500 mg niacin or its derivatives co-packaged to provide a dosage up to 3000 mg of niacin. The present invention includes a kit for multiple unit dosage form comprising one capsule of 500 mg and one capsule of 250 mg niacin or its derivatives co-packaged to provide a dosage of 750 mg of niacin. The present invention also includes a kit for multiple unit dosage form comprises of two capsules of 500 mg niacin or its derivatives co-packaged to provide a dosage of 1000 mg of niacin.

A kit includes one or more multiple unit dosage form packaged to visually and/or tactilely indicate an appropriate sequence for administering anti-hyperlipidemic agent(s), more preferably niacin or its derivatives and optionally HMG CoA reductase inhibitor.

The kit of the present invention contains a combination of one or more multiple unit dosage form comprising niacin or its derivatives.

The kit of the present invention may further optionally contain a combination of one or more multiple unit dosage form comprising HMG CoA reductase inhibitor.

The kits are packaged to provide appropriate dosage of niacin or its derivatives in a convenient form for administration. In one embodiment, the kit contains multiple solid oral dosage forms in the same or different dosages.

The packaging material may be a box, bottle, pouch, blister package, strip package tray, or card. Any other package, which comprises of one or more multiple unit dosage form, is included within the scope of the invention. The kit typically may also include instructions for coordinating the administration of anti-hyperlipidemic agent(s), most preferably niacin or its derivatives and HMG CoA reductase inhibitors.

The package may comprise of one or more multiple unit dosage form of niacin or its derivatives and HMG CoA reductase inhibitors, separately or in combination arranged for sequential or simultaneous administration.

Another embodiment of the present invention includes the method of manufacturing the multiple unit comprising at least or more than about 50% of niacin and control releasing agent to be filled in capsule dosage form, optionally comprising HMG CoA reductase inhibitors. Preferably the present invention involves the multiple unit dosage form of niacin comprising niacin and pharmaceutically acceptable excipients coated with control releasing agent. The HMG CoA reductase inhibitor may be in the form of controlled release or immediate release form.

The process for manufacturing the multiple unit dosage form of the present invention is not limited to the processes described in the application and the dosage form can be prepared by using any of the processes known to one skilled in the art. The process for manufacturing the multiple unit dosage form of niacin or its derivatives and HMG CoA reductase inhibitor can be prepared together or separately. Anti-hyperlipidemic agent(s) along with or without directly compressible grade excipients or granulated together or separately by wet granulation or dry granulation with or without excipients. The granules of anti-hyperlipidemic agent(s) are prepared by sifting the anti-hyperlipidemic agent(s) and excipients through the desired mesh size sieve and then are mixed together or separately using a rapid mixer granulator or planetary mixer or mass mixer or ribbon mixer or fluid bed processor or any other suitable device. The blend can be granulated together or separately by adding a solution of a binder whether alcoholic or hydroalcoholic or aqueous in a low or high shear mixer, fluidized bed granulator and the like or by dry granulation. The granulate can be dried together or separately by using a tray drier or fluid bed drier or rotary cone vacuum drier and the like. The sizing of the granules can be done using an oscillating granulator or comminuting mill or any other conventional equipment equipped with a suitable screen. Alternatively, granules or pellets or spheroids can be prepared by extrusion and spheronization, or roller compaction. Also the manufacture of granules of anti-hyperlipidemic agent(s) can be made by mixing the directly compressible excipients or by roller compaction. The blend so obtained can be compressed using a suitable device, such as a station rotary machine to form slugs, which are passed through a mill or fluid energy mill or ball mill or colloid mill or roller mill or hammer mill and the like, equipped with a suitable screen to obtain the milled slugs. In another aspect of the invention, the smaller tablets (mini-tablets) can be made by compressing the granules, pellets, spheroids, particles using die-and-punch of various sizes and shapes, as desired. The multiple unit dosage form of the present invention may further be coated. The multiple unit dosage form can be coated using control releasing polymer or film forming polymer or combination thereof. The multiple unit dosage form of niacin or its derivatives and HMG CoA reductase inhibitor can be coated together or separately. Further the HMG CoA reductase inhibitor may be present in the coating on the multiple unit of niacin or its derivatives. One or more coating layers are also possible. The amount of coating used depends upon the rate of release required. Preferably the coating is applied till the weight gain is about 2 to about 20%. More preferably the coating is applied till a weight gain of about 5 to about 15%. Optionally, the coating on the powder, granules, pellets, spheroids, particles, minitablets can be applied by techniques known to one skilled in the art such as spray coating, dip coating, fluidized bed coating and the like.

The coating solution may include control releasing agent or film forming agent and other pharmaceutically acceptable excipients including but not limiting to plasticizer, dispersants, solvent and colorants.

Plasticizer is selected from the group but not limited to polyglyceryl fatty acid esters, polyoxyethylene glyceryl fatty acid esters, sorbitan fatty acid esters, poly-oxyethylene sorbitol fatty acid esters, polyethylene glycol and polyethylene glycol fatty acid esters, polyoxyethylene castor oils, polyoxyethylene alkyl ethers, polyoxyethylene phytosterols, polyoxyethylene alkyl phenyl ethers, polyoxyethylene polyoxypropylene alkyl ethers, polyoxyethylene lanolines, polyoxyethylene alkyl ether phosphates, propylene glycol and propylene glycol fatty acids, glycerol fatty acid esters, fatty acid macrogol glycerides, glyceryl monolinoleate, glyceryl monooleate, diethylene glycol monoethyl ether, benzylbenzoate, chlorobutanol, dibutyl sebacate, diethyl phthalate, glycerin, mineral oil and lanolin alcohol, petroleum and lanolin alcohol, triacetin and triethyl citrate, sodium lauryl sulfate, docusate sodium, sorbitol and mannitol.

Dispersant is selected from the group but not limited to talc, precipitated calcium carbonate, magnesium stearate, calcium sulfate, sugar, microsilica and calcium phosphate, starches.

Colorant is selected from the group but not limited to titanium oxide, zinc oxide, soluble and tar pigment.

Solvent for coating material may be organic or aqueous or combination thereof.

The capsule may further be polished using polishing agents including but not limited to carnauba wax and bees wax.

The following examples are illustrative of the present invention, and the example should not be considered as limiting the scope of this invention in any way, as these examples and other equivalents thereof will become apparent to those versed in the art, in the light of the present disclosure, and the accompanying claims.

### Example 1

| Sr. No. | Ingredients | Percentage |
|---|---|---|
| 1 | Niacin | 80 |
| 2 | Microcrystalline cellulose | 8 |
| 3 | Hydroxypropyl methylcellulose | 0.8 |

Coating

| | | |
|---|---|---|
| 1 | Hydroxypropyl methyl cellulose | 2.74 |
| 2 | Ethyl Cellulose | 6.45 |
| 3 | Triethyl citrate | 0.6 |
| 4 | Talc | 0.6 |
| 5 | Dichloromethane/IPA | q.s. |

### Procedure:

Weighed and mixed the defined quantities of the sieved active ingredient along with microcrystalline cellulose. The blend was granulated using a solution of hydroxypropyl methylcellulose in water and the wet mass was passed through an extruder. The extruded mass was processed in an spheronizer to obtain pellets. The obtained pellets were dried. The pellets are further coated with rate controlling coating solution for controlled release of the drug. The coated pellets were filled inside a capsule.

### Dissolution of Multiple Unit Dosage Form:

The dissolution profile of the formulation of the present invention is as below:

### Dissolution Profile:

Medium—water 1000 mL, RPM—100

| Sr No. | Time (hours) | Drug release (%) |
|---|---|---|
| 1 | 4 | 10%-45% |
| 2 | 8 | 35%-75% |
| 3 | 12 | 65%-80% |
| 4 | 16 | 75%-85% |
| 5 | 20 | 80%-90% |
| 6 | 24 | NLT 95% |

### Example 2

| Sr. No. | Ingredients | Percentage |
|---|---|---|
| 1 | Niacin | 73 |
| 3 | Polyethylene oxide | 16 |
| 4 | Hydroxypropyl methyl cellulose | 1.3 |
| 5 | IPA | q.s. |

Coating

| | | |
|---|---|---|
| 1 | Hydroxypropyl methyl cellulose | 2.5 |
| 2 | Ethyl Cellulose | 6 |
| 3 | Triethyl citrate | 0.6 |
| 4 | Talc | 0.6 |
| 5 | Dichloromethane/IPA | q.s. |

### Procedure:

Weighed and mixed the defined quantities of the sieved active ingredient along with polyethylene oxide, hydroxypropyl methylcellulose. The blend was granulated using isopropyl alcohol and the wet mass was passed through an extruder. The extruded mass was processed in an spheronizer to obtain pellets. The obtained pellets were dried. The pellets are further coated with rate controlling coating solution for controlled release of the drug. The coated pellets were filled inside a capsule.

### Example 3

| Sr. No. | Ingredients | Percentage |
|---|---|---|
| 1 | Niacin | 50 |
| 2 | Microcrystalline cellulose | 5 |
| 3 | Crospovidone | 5 |
| 4 | Hydroxypropyl methyl cellulose | 3 |
| 5 | Talc | 1 |
| 6 | Magnesium stearate | 1 |

Coating

| | | |
|---|---|---|
| 1 | Hydroxypropyl methyl cellulose | 10 |
| 2 | Ethyl Cellulose | 15 |
| 3 | Triethyl citrate | 5 |
| 4 | Talc | 5 |
| 5 | Dichloromethane/IPA | q.s. |

### Procedure:

Weighed quantity of the active ingredient, filler or diluent and disintegrant was granulated with the binder preparation, sieved and lubricated using lubricant. The prepared blend was compressed into mini-tablet using a rotary compression machine. These uncoated mini-tablets were further coated with controlled release coating solution. The coated mini-tablets were further filled into capsule.

### Example 4

| Sr. No. | Ingredients | Percentage |
|---|---|---|
| 1 | Niacin | 78 |
| 2 | Hydroxypropyl methylcellulose | 5 |
| 3 | Ethyl cellulose | 15 |
| 4 | Triethyl citrate | 1 |
| 5 | Talc | 1 |
| 6 | Dichloromethane/IPA | q.s. |

### Procedure:

Weighed quantity of the active ingredient was passed through sieve and placed in the bottom bowl of fluidized bed coater. Coating solution was prepared by dispersing hydroxypropyl methylcellulose and ethyl cellulose, triethyl citrate and talc in suitable solvent system. The active ingredient was further coated with rate controlling coating solution using fluidized bed coater.

### Example 5

| Sr. No. | Ingredients | Percentage |
|---|---|---|
| 1 | Niacin | 71 |
| 2 | Microcrystalline cellulose | 7 |
| 3 | Povidone | 0.7 |
| 4 | Purified water | q.s |

Coating

| | | |
|---|---|---|
| 1 | Eudragit | 10 |
| 2 | Talc | 10 |
| 3 | Triethyl citrate | 1.5 |
| 4 | Sodium hydroxide | 0.12 |
| 5 | Purified water | q.s. |

### Procedure:

Weighed and mixed the defined quantities of the sieved active ingredient along with microcrystalline cellulose and povidone. The blend was granulated using purified water and the wet mass was passed through an extruder. The extruded mass was processed in an spheronizer to obtain pellets. The obtained pellets were dried. Coating solution was prepared by dispersing eudragit, talc and triethyl citrate in purified water. The pellets were further coated with above aqueous dispersion for controlled release of the drug. The coated pellets were filled inside a capsule.

### Example 6

| | | |
|---|---|---|
| Sr. No. | Ingredients | Percentage |

Part A - Niacin

| | | |
|---|---|---|
| 1 | Niacin | 74 |
| 2 | Microcrystalline cellulose | 8 |
| 3 | Hydroxypropyl methylcellulose | 0.8 |

Coating

| | | |
|---|---|---|
| 1 | Hydroxypropyl methyl cellulose | 2.74 |
| 2 | Ethyl Cellulose | 6.45 |
| 3 | Triethyl citrate | 0.6 |
| 4 | Talc | 0.6 |
| 5 | Dichloromethane/IPA | q.s. |

Part B - Lovastatin

| | | |
|---|---|---|
| 1 | Lovastatin | 2.9 |
| 2 | Polyvinyl pyrrolidone | 1.51 |
| 3 | Colloidal silicone dioxide | 1.2 |
| 4 | Magnesium stearate | 1.2 |

### Procedure:

### Part A—Niacin

Weighed and mixed the defined quantities of the sieved active ingredient along with microcrystalline cellulose. The blend was granulated using a solution of hydroxypropyl methylcellulose in water and the wet mass was passed through an extruder. The extruded mass was processed in an spheronizer to obtain pellets. The obtained pellets were dried. The pellets are further coated with rate controlling coating solution for controlled release of the drug.

### Part B—Lovastatin

Weighed and mixed the defined quantities of the sieved active ingredient along with colloidal silicon dioxide. The blend was granulated using a solution of polyvinyl pyrrolidone in water and the wet mass was passed through an extruder. The extruded mass was processed in an spheronizer to obtain pellets. The obtained pellets were dried. The coated pellets of niacin and uncoated pellets of lovastatin were filled inside a capsule.

### Example 7

| | | |
|---|---|---|
| Sr. No. | Ingredients | Percentage |

Part A - Niacin

| | | |
|---|---|---|
| 1 | Niacin | 70 |
| 2 | Hydroxypropyl methylcellulose | 6.19 |

Coating

| | | |
|---|---|---|
| 1 | Ethyl cellulose | 15 |
| 2 | Triethyl citrate | 1 |
| 3 | Talc | 1 |
| 4 | Dichloromethane/IPA | q.s. |

Part B - Lovastatin

| | | |
|---|---|---|
| 1 | Lovastatin | 2.9 |
| 2 | Polyvinyl pyrrolidone | 1.51 |
| 3 | Colloidal silicone dioxide | 1.2 |
| 4 | Magnesium stearate | 1.2 |

### Procedure:

### Part A—Niacin

Weighed quantity of the active ingredient was passed through sieve and placed in the bottom bowl of fluidized bed coater. Coating solution was prepared by dispersing hydroxypropyl methylcellulose and ethyl cellulose, triethyl citrate and talc in suitable solvent system. The active ingredient was further coated with rate controlling coating solution using fluidized bed coater.

### Part B—Lovastatin

Weighed and mixed the defined quantities of the sieved active ingredient along with colloidal silicon dioxide. The blend was granulated using a solution of polyvinyl pyrrolidone in water and the wet mass was passed through an extruder. The extruded mass was processed in an spheronizer to obtain pellets. The obtained pellets were dried. The coated particles of niacin and uncoated pellets of lovastatin were filled inside a capsule.

The multiple unit dosage form of niacin may also be prepared by mixing the above mentioned examples in different proportions so that proper dose of the niacin can be administered in a controlled manner. A person skilled in the art may make variations and modifications without deviating from the spirit and scope of the invention. All such modifications and variations are intended to be included within the scope of the invention.

1. A multiple unit dosage form comprising at least or more than about 50% of a therapeutically effective amount of antihyperlipidemic agent and one or more control releasing agent(s) and pharmaceutically acceptable excipients, weight percentages are based upon the total weight of the dosage form.

2. A multiple unit dosage form according to claim 1, wherein the multiple unit dosage form is capsule.

3. A multiple unit dosage form according to claim 1, wherein antihyperlipidemic agent is niacin or its derivatives.

4. A multiple unit dosage form according to claim 1, wherein the multiple unit dosage form comprises at least or more than about 65% of niacin or its derivatives.

5. A multiple unit dosage form according to claim 1, wherein control-releasing agent is selected from the group comprising hydrophilic agent or hydrophobic agent or combination thereof.

6. A multiple unit dosage form according to claim 5, wherein hydrophilic agent is selected from the group but not limited to cellulose derivatives; saccharide family, alginic acid alkali metal salt as a family of alginic acid; gum family, peptide series; acrylic acid series carbomer, polyacrylamide, and other materials (e.g., polyvinylacetal diethylamino acetate, glucomannan, glucosamine, arabinogalactane, furcelleran, pullulan, polyurethane, chitin family, agar, pectin and carrageenan, polyvinyl acetate, polyvinyl alcohol, povidone, polyethylene oxide and mixtures thereof.

7. A multiple unit dosage form according to claim 5, wherein hydrophobic agent is selected from the group but not limited to water-insoluble cellulose derivatives, water-insoluble vinyl derivatives, paraffin and stearate salts, acrylic acid and methacrylic acid copolymers, waxes and mixture thereof.

8. A multiple unit dosage form according to claim 1, wherein the multiple unit dosage form further comprise of HMG CoA reductase inhibitor.

9. A multiple unit dosage form according to claim 8, wherein HMG CoA reductase inhibitor is selected from mevastatin, lovastatin, pitavastatin, velostatin, simvastatin, rivastatin, fluvastatin, atorvastatin and cerivastatin.

10. The multiple unit dosage form comprising at least or more than about 50% of a therapeutically effective amount of niacin or its derivatives and control releasing agent(s), wherein the control releasing agent is used in combination of hydrophobic and hydrophilic agent in the ratio of about 1:50 to 50:1, weight percentages are based upon the total weight of the dosage form.

11. A multiple unit dosage form according to claim 10, wherein the multiple unit dosage form is capsule.

12. The multiple unit dosage form according to claim 10, wherein the control-releasing agent is used in combination of hydrophobic and hydrophilic agent more preferably in the ratio of about 1:25 to 25:1.

13. A multiple unit dosage form according to claim 12, wherein hydrophilic agent is selected from the group but not limited to cellulose derivatives; saccharide family, alginic acid alkali metal salt as a family of alginic acid; gum family, peptide series; acrylic acid series carbomer, polyacrylamide, and other materials (e.g., polyvinylacetal diethylamino acetate, glucomannan, glucosamine, arabinogalactane, furcelleran, pullulan, polyurethane, chitin family, agar, pectin and carrageenan, polyvinyl acetate, polyvinyl alcohol, povidone, polyethylene oxide and mixtures thereof.

14. A multiple unit dosage form according to claim 12, wherein hydrophobic agent is selected from the group but not limited to water-insoluble cellulose derivatives, water-insoluble vinyl derivatives, paraffin and stearate salts, acrylic acid and methacrylic acid copolymers, waxes and mixture thereof.

15. A multiple unit dosage form according to claim 10, wherein the multiple unit dosage form further comprise of HMG CoA reductase inhibitor.

16. A multiple unit dosage form according to claim 15, wherein HMG CoA reductase inhibitor is selected from mevastatin, lovastatin, pitavastatin, velostatin, simvastatin, rivastatin, fluvastatin, atorvastatin and cerivastatin

17. A multiple unit dosage form comprising at least or more than about 65% of a therapeutically effective amount of niacin or its derivatives, one or more control releasing agent(s) filled in capsule of suitable size, weight percentages are based upon the total weight of the dosage form.

18. A multiple unit dosage form according to claim 17, wherein control-releasing agent is selected from the group comprising hydrophilic agent or hydrophobic agent or combination thereof.

19. A multiple unit dosage form according to claim 18, wherein hydrophilic agent is selected from the group but not limited to cellulose derivatives; saccharide family, alginic acid alkali metal salt as a family of alginic acid; gum family, peptide series; acrylic acid series carbomer, polyacrylamide, and other materials (e.g., polyvinylacetal diethylamino acetate, glucomannan, glucosamine, arabinogalactane, furcelleran, pullulan, polyurethane, chitin family, agar, pectin and carrageenan, polyvinyl acetate, polyvinyl alcohol, povidone, polyethylene oxide and mixtures thereof.

20. A multiple unit dosage form according to claim 18, wherein hydrophobic agent is selected from the group but not limited to water-insoluble cellulose derivative, water-insoluble vinyl derivative, paraffin and stearate salts, acrylic acid and methacrylic acid copolymers, waxes and mixture thereof.

21. A multiple unit dosage form according to claim 17, wherein the multiple unit dosage form further comprise of HMG CoA reductase inhibitor.

22. A multiple unit dosage form according to claim 21, wherein HMG CoA reductase inhibitor is selected from mevastatin, lovastatin, pitavastatin, velostatin, simvastatin, rivastatin, fluvastatin, rosuvastatin, atorvastatin and cerivastatin

23. A multiple unit dosage form comprising at least or more than about 65% of a therapeutically effective amount of niacin or its derivatives coated with one or more control release agent(s) wherein the percent weight gain is from about 2 to about 20%, filled in capsule of suitable size, weight percentages are based upon the total weight of the dosage form.

24. A multiple unit dosage form according to 23, wherein the weight gains in coating is about 5 to about 15%.

25. A multiple unit dosage form according to claim 23, wherein control-releasing agent is selected from the group comprising hydrophilic agent or hydrophobic agent or combination thereof.

26. A multiple unit dosage form according to claim 25, wherein hydrophilic agent is selected from the group but not limited to cellulose derivatives; saccharide family, alginic acid alkali metal salt as a family of alginic acid; gum family, peptide series; acrylic acid series carbomer, polyacrylamide, and other materials (e.g., polyvinylacetal diethylamino acetate, glucomannan, glucosamine, arabinogalactane, furcelleran, pullulan, polyurethane, chitin family, agar, pectin and carrageenan, polyvinyl acetate, polyvinyl alcohol, povidone, polyethylene oxide and mixtures thereof.

27. A multiple unit dosage form according to claim 25, wherein hydrophobic agent is selected from the group but not limited to water-insoluble cellulose derivative, water-insoluble vinyl derivative, paraffin and stearate salts, acrylic acid and methacrylic acid copolymers, waxes and mixture thereof.

28. A multiple unit dosage form according to claim 23, wherein the multiple unit dosage form further comprise of HMG CoA reductase inhibitor.

29. A multiple unit dosage form according to claim 28, wherein HMG CoA reductase inhibitor is selected from mevastatin, lovastatin, pitavastatin, velostatin, simvastatin, rivastatin, fluvastatin, rosuvastatin, atorvastatin and cerivastatin

30. A kit for multiple unit dosage form of niacin or its derivatives capsule comprising one or more capsule co-packaged to provide a dosage up to 2000 mg of niacin.

31. A kit for multiple unit dosage form according to claim 30, comprising two or more capsules of 500 mg niacin or its derivatives co-packaged to provide a dosage up to 2000 mg of niacin.

32. A kit for multiple unit dosage form according to claim 30, comprising one capsule of 500 mg and one capsule of 250 mg niacin or its derivatives co-packaged to provide a dosage of 750 mg of niacin.

33. A kit for multiple unit dosage form according to claim 30, comprising two capsules of 500 mg niacin or its derivatives co-packaged to provide a dosage of 1000 mg of niacin.

34. A kit for multiple unit dosage form according to claim 30, further comprises multiple unit of HMG CoA reductase inhibitor.

35. A multiple unit dosage form comprising control release niacin or its derivatives in combination with HMG CoA reducatse inhibitor filled in capsule form.

36. A multiple unit dosage form according to claim 35, wherein HMG CoA reductase inhibitor is selected from mevastatin, lovastatin, pitavastatin, velostatin, simvastatin, rivastatin, fluvastatin, rosuvastatin, atorvastatin and cerivastatin

37. A multiple unit dosage form according to claim 36, wherein HMG CoA reductase inhibitor is more preferably in immediate release form.
